(19) United States
(12) Patent Application Publication
(10) Pub. No.: US 2025/0143618 A1
(43) Pub. Date: May 8, 2025

(54) ELECTROCARDIOGRAM ANALOG TO DIGITAL CONVERTER

(71) Applicant: Bardy Diagnostics, Inc., Bellevue, WA (US)

(72) Inventors: Earl Herleikson, Marion, MT (US); Yunqiang Lin, Bellevue, WA (US)

(21) Appl. No.: 18/925,506

(22) Filed: Oct. 24, 2024

Related U.S. Application Data
(60) Provisional application No. 63/595,578, filed on Nov. 2, 2023.

(51) Int. Cl. A61B 5/308; A61B 5/00

(52) U.S. Cl. CPC A61B 5/308; A61B 5/7225; A61B 2560/0214

(57) ABSTRACT

An analog to digital converter for use in an electrocardiogram device is provided that includes a voltage supply; an operational amplifier, including: a positive input pin; an inverting input pin; and an output pin; an output node, connected directly to the output pin and the inverting input pin; an ECG electrode, connected to the output node via a first resistor; a reference voltage, connected directly to the output node; and a feedback node, connected to directly to the positive input pin, connected to the voltage supply via a second resistor, and connected to a ground via a third resistor and a first capacitor, wherein the third resistor and the first capacitor are connected in parallel between the feedback node and the ground.

### CROSS-REFERENCES TO RELATED DISCLOSURES

The present disclosure claims the benefit of and priority to U.S. Provisional Patent Application No. 63/595,578 titled “ELECTROCARDIOGRAM ANALOG TO DIGITAL CONVERTER” and filed on Nov. 2, 2023, which is incorporated herein it its entirety.

### FIELD

The present disclosure generally relates to medical tools, and in particular to an analog to digital converter (ADC) for use in electrocardiogram (ECG) signal gathering.

### BACKGROUND

Electrocardiograms (ECGs, also referred to as EKGs) are recordings of the electrical signals of a biological subject's heart. Electrocardiogrameasures the electrical activity of the cardiac cycle; graphing the voltage over time due to the muscle depolarization and repolarization that occurs during a heartbeat. Various medical conditions can be detected through the analysis of ECGs to identify shifts in rhythm, magnitude, or waveform shape from a baseline for the biological subject. An ECG in a human biological subject is measured from a baseline voltage and includes a series of waves (e.g., P-wave, Q-wave) that can be used to diagnose various cardiac conditions based on the relative magnitudes and timings in one heartbeat or across (whether consistently or sporadically) across several heartbeats.

### SUMMARY

The present disclosure provides enhanced electrocardiogram (ECG) analog to digital converters (ADCs). An ECG measurement patch adhered to the skin for the longitudinal analysis of a biological subject's ECG over an extended duration (e.g., a month or more) needs to be powered over the length of analysis period, which may require the replacement, recharging, or conservation of battery power in the device. As these devices are adhered to the skin of the biological subject, design goals frequently prioritize reducing power consumption, which reduces the size and weight of the needed battery and avoids or mitigates the need to replace or recharge the battery, which in turn reduces the risk of an operator forgetting to replace/recharge the battery, improves adhesion to the biological subject's skin, reduces discomfort of wearing the devices, and various other benefits. Accordingly, the described devices are smaller and lighter and have a power source that can last over a longer duration that previous devices, among other benefits offered.

The design discussed in the present disclosure also provides an increased signal to noise ratio (SNR), which when using a small ECG spacing between electrodes (having smaller signal amplitude than larger spacing arrangements) allows the ECG system to more readily identify P-waves in the ECG for accurate diagnosis.

The design discussed in the present disclosure also provides a high dynamic range for ECG measurements. In various embodiments, the measurement of the ST segment in the ECG is performed with a Direct Current (DC) coupled ADC with ±300 millivolts (mV) of range and 1 microvolt (μV) least significant bit (LSB). The dynamic range of the present design allows for compensations against baseline wander in the ECGs, which can be caused when subjects wear a patch for longer periods of time, and can drive the ECG to the edges of the ECG measurement range and clip or distort the ECG signal if not accounted for.

In one embodiment, an analog to digital (A2D) converter for an electrocardiogram (ECG) measuring device is provided, comprising: a voltage supply; an operational amplifier, including: a positive input pin; an inverting input pin; and an output pin; an output node, connected directly to the output pin and the inverting input pin; an ECG electrode, connected to the output node via a first resistor; a reference voltage, connected directly to the output node; and a feedback node, connected to directly to the positive input pin, connected to the voltage supply via a second resistor, and connected to a ground via a third resistor and a first capacitor, wherein the third resistor and the first capacitor are connected in parallel between the feedback node and the ground.

In some embodiments, the A2D converter further comprises a self-test electrode connected to the first resistor via a fifth resistor.

In some embodiments, the operational amplifier further includes a supply voltage pin, a shutdown pin, and a ground pin that is connected to the ground, further comprising: a voltage supply, connected directly to the supply voltage pin and connected to the shutdown pin via a second resistor.

In some embodiments, the A2D converter of claim 1, further comprises: a second operational amplifier, including: a second positive input pin; a second inverting input pin; and a second output pin; a second output node, connected directly between a fifth resistor connected to the second output pin and connected to a second capacitor that is connected to the reference voltage; a second ECG electrode, connected to the second positive input pin via at least one resistor; a third capacitor connected to the second positive input pin and the reference voltage; a sixth resistor and a fourth capacitor connected in series between the reference voltage and the second inverting input pin; a fifth capacitor connected between the second inverting input pin and the second output pin; and a Pulse Wave Modulated (PWM) signal source connected to the second inverting input pin.

In one embodiment, an analog to digital (A2D) converter for an electrocardiogram (ECG) measuring device is provided, comprising: an operational amplifier, including: a positive input pin; an inverting input pin; and an output pin; an output node, connected directly between a first resistor connected to the output pin and connected to a first capacitor that is connected to a reference voltage; an ECG electrode, connected to the positive input pin via at least one resistor; a second capacitor connected to the positive input pin and the reference voltage; a second resistor and a third capacitor connected in series between the reference voltage and the inverting input pin; a fourth capacitor connected between the inverting input pin and the output pin; and a Pulse Wave Modulated (PWM) signal source connected to the inverting input pin.

In some embodiments, the operational amplifier further includes a supply voltage pin, a shutdown pin, and a ground pin that is connected to a ground, further comprising: a voltage supply, connected directly to the supply voltage pin and connected to the shutdown pin via a second resistor.

In some embodiments, the at least one resistor and the second capacitor define a low pass filter with a corner frequency of approximately 1.6 kilohertz.

In some embodiments, the second resistor and the third capacitor define a low pass filter with a corner frequency of approximately 81 Hertz.

In some embodiments, the first resistor and the first capacitor define a low pass filter with a corner frequency of approximately 1 kilohertz.

In some embodiments, the PWM signal source combines a first digital PWM signal with a second digital PWM signal before delivering a combined PWM signal to the inverting input pin.

In some embodiments, the A2D converter further comprises: a voltage supply; a second operational amplifier, including: a second positive input pin; a second inverting input pin; and a second output pin; an output node, connected directly to the output pin and the inverting input pin; a second ECG electrode, connected to the output node via a third resistor; a reference electrode, connected directly to the output node; a feedback node, connected to directly to the positive input pin, connected to the voltage supply via a third resistor, and connected to a ground via a fourth resistor and a first capacitor, wherein the fourth resistor and the first capacitor are connected in parallel between the feedback node and the ground.

In some embodiments, the A2D converter further comprises a self-test electrode connected to the first resistor via a fifth resistor.

In one embodiment, an analog to digital (A2D) converter for an electrocardiogram (ECG) measuring device is provided, comprising: an operational amplifier; a first low pass filter, connected between an ECG electrode, a reference voltage, and a positive input pin of the operational amplifier; a second low pass filter, connected between the reference voltage and an inverting input of the operational amplifier; a third low pass filter, connected between an output pin of the operational amplifier, the reference voltage, and a signal output; a first signal pathway connected between a first digital pulse wave modulated (PWM) signal source and the inverting input of the operational amplifier; and a second signal pathway connected between a second digital PWM signal source and the inverting input of the operational amplifier, wherein the second signal pathway combines with the first signal pathway before connecting with the first signal pathway.

In some embodiments, the first low pass filter has a corner frequency of approximately 1.6 kilohertz and includes a series of resistors disposed between the ECG electrode and the positive input pin and a capacitor disposed between the positive input pin and the reference voltage.

In some embodiments, the second low pass filter has a corner frequency of approximately 81 Hertz and includes a resistor connected between the reference voltage and a capacitor, wherein the capacitor is connected between the resistor and the inverting input of the operational amplifier.

In some embodiments, the third low pass filter has a corner frequency of approximately 1 kilohertz and includes a resistor connected between the output pin of the operational amplifier and the signal output and include a capacitor is connected between the signal output and the reference voltage.

In some embodiments, the first signal pathway has a first resistance, and the second signal pathway has a second resistance, greater than the first resistance, as measured between respective pins of a driver chip and a node shared with the inverting input of the operational amplifier.

In some embodiments, the A2D converter further comprises: a voltage supply, connected directly to a supply voltage pin of the operational amplifier and connected to a shutdown pin of the operational amplifier via a resistor, transistor, or switch.

In some embodiments, the A2D converter further comprises: a second operational amplifier, including: a second positive input pin; a second inverting input pin; and a second output pin; a second output node, connected directly to the second output pin and the second inverting input pin; a second ECG electrode, connected to the second output node via a first resistor; a reference electrode, connected directly to the second output node; a feedback node, connected directly to the second positive input pin, connected to the voltage supply via a second resistor, and connected to the ground via a third resistor and a first capacitor, wherein the third resistor and the first capacitor are connected in parallel between the feedback node and the ground.

In some embodiments, the A2D converter further comprises a self-test electrode connected to the first resistor via a fifth resistor.

Still other embodiments will become readily apparent to those skilled in the art from the following detailed description, wherein are described embodiments by way of illustrating the best mode contemplated. As will be realized, other and different embodiments are possible and the embodiments' several details are capable of modifications in various obvious respects, including time and clustering of events, all without departing from their spirit and the scope. Accordingly, the drawings and detailed description are to be regarded as illustrative in nature and not as restrictive.

### BRIEF DESCRIPTION OF THE DRAWINGS

FIG. 1 illustrates an example of an electrocardiogramonitor, including an extended wear electrode patch fitted to the sternal region of a human male biological subject, according to embodiments of the present disclosure.

FIG. 2 is a circuit diagram of an example patch connection for an electrocardiogram (ECG) reader, according to embodiments of the present disclosure.

FIGS. 3A and 3B are circuit diagrams of an example patch connection for an ECG reader with an Analog to Digital Converter (ADC) therein, according to embodiments of the present disclosure.

FIGS. 4A and 4B illustrate digital signals used by the ADC described herein for measuring an ECG waveform, according to embodiments of the present disclosure.

FIG. 5 illustrates an example computing device, according to embodiments of the present disclosure.

### DETAILED DESCRIPTION

The present disclosure provides enhanced electrocardiogram (ECG) analog to digital converters (ADCs, also referred to as A2D converters). An ECG measurement patch adhered to the skin for the longitudinal analysis of a biological subject's ECG over an extended duration (e.g., a month or more) needs to be powered over the length of analysis period, which may require the replacement, recharging, or conservation of battery power in the device. As these devices are adhered to the skin of the biological subject, design goals frequently prioritize reducing power consumption, which reduces the size and weight of the needed battery and avoids or mitigates the need to replace or recharge the battery, which in turn reduces the risk of an operator forgetting to replace/recharge the battery, improves adhesion to the biological subject's skin, reduces discomfort of wearing the devices, and various other benefits. Accordingly, the described devices are smaller and lighter and have a power source that can last over a longer duration that previous devices, among other benefits offered.

The design discussed in the present disclosure also provides an increased signal to noise ratio (SNR), which when using a small ECG spacing between electrodes (having smaller signal amplitude than larger spacing arrangements) allows the ECG system to more readily identify P-waves in the ECG for accurate diagnosis.

The design discussed in the present disclosure also provides a high dynamic range for ECG measurements. In various embodiments, the measurement of the ST segment in the ECG is performed with a Direct Current (DC) coupled ADC with ±300 millivolts (mV) of range and 1 microvolt (μV) least significant bit (LSB). The dynamic range of the present design allows for compensations against baseline wander in the ECGs, which can be caused when subjects wear a patch for longer periods of time, and can drive the ECG to the edges of the ECG measurement range and clip or distort the ECG signal if not accounted for.

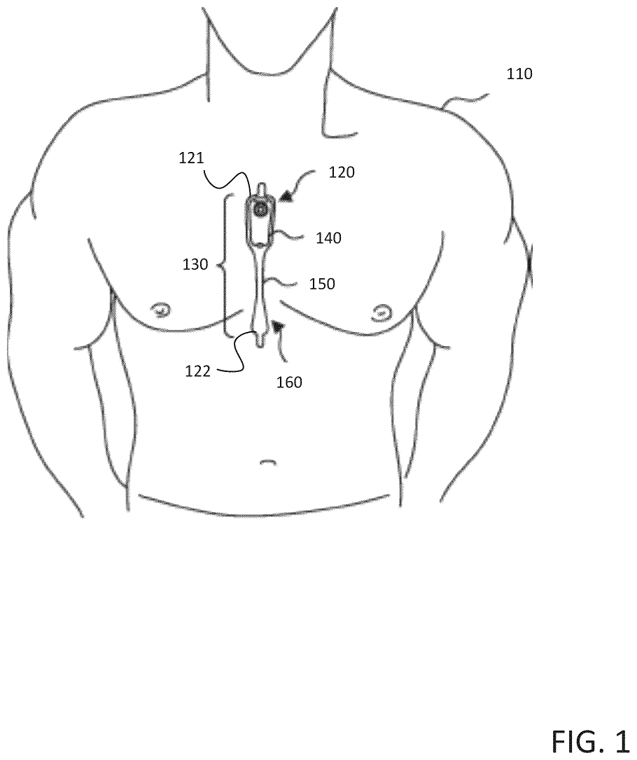

FIG. 1 illustrates an example of an electrocardiogramonitor, including an extended wear electrode patch fitted to the sternal region of a human male biological subject, according to embodiments of the present disclosure. ECG and physiological monitoring can be provided through a wearable ambulatory monitor that includes two components: a flexible extended wear electrode patch and a removable reusable (or single use) monitor recorder. Both the electrode patch and the monitor recorder are configured to capture electrical signals from the propagation of low amplitude, relatively low frequency content cardiac action potentials, such as the P-waves generated during atrial activation. By way of example, FIG. 1 shows an extended wear electrocardiogramonitor 120, including a monitor recorder 140. The wearable monitor 12 sits centrally, positioned axially along the sternal midline 160, on the subject's chest along the sternum 130 and oriented top-to-bottom with the monitor recorder 140 situated (preferably) towards the subject's head.

The electrode patch 150 is shaped to fit comfortably and conformal to the contours of the subject's chest approximately centered on the sternal midline 160 (or immediately to either side of the sternum 130). The distal end of the electrode patch 150, under which a lower or inferior pole 122 (ECG electrode) is adhered, extends towards the Xiphoid process and lower sternum, and (depending upon the subject's build) may straddle the region over the Xiphoid process and lower sternum. The proximal end of the electrode patch 150, located under the monitor recorder 140, under which an upper or superior pole 121 (ECG electrode) is adhered, is below the manubrium and, depending upon subject's build, may straddle the region over the manubrium.

Although discussed herein primarily with reference to a biological subject that is human, the present disclosure contemplates that the teachings provided herein may be applied for electrocardiogramith non-human biological subjects. Accordingly, the various anatomical terms used to describe physiological features and relative locations in a human model may be modified to describe recognizably similar physiological features and relative locations in non-human models.

During ECG monitoring, the amplitude and strength of action potentials sensed on the body's surface are affected to varying degrees by cardiac, cellular, extracellular, vector of current flow, and physical factors, like obesity, dermatitis, high impedance skin, etc. Sensing along the sternal midline 160 (or immediately to either side of the sternum 130) significantly improves the ability of the wearable monitor 120 to cutaneously sense cardiac electric signals, particularly the P-wave (or atrial activity) and, to a lesser extent, the QRS interval signals in the ECG waveforms that indicate ventricular activity by countering some of the effects of these factors. Depending upon the placement locations on the chest, ECG electrodes may be separated from activation regions within the heart by differing combinations of internal tissues and body structures, including heart muscle, intracardiac blood, the pericardium, intrathoracic blood and fluids, the lungs, skeletal muscle, bone structure, subcutaneous fat, and the skin, plus any contaminants present between the skin's surface and electrode signal pickups. The degree of amplitude degradation of cardiac transmembrane potentials increases with the number of tissue boundaries between the heart and the skin's surface that are encountered. The cardiac electrical field is degraded each time the transmembrane potentials encounter a physical boundary separating adjoining tissues due to differences in the respective tissues' electrical resistances. In addition, other non-spatial factors, such as pericardial effusion, emphysema, or fluid accumulation in the lungs, can further degrade body surface potentials.

These degradations in the heartbeat signal due to effects from the physiology of the biological subject all decrease the Signal to Noise Ratio (SNR) in generating an ECG; either by increasing noise magnitude or decreasing signal magnitude.

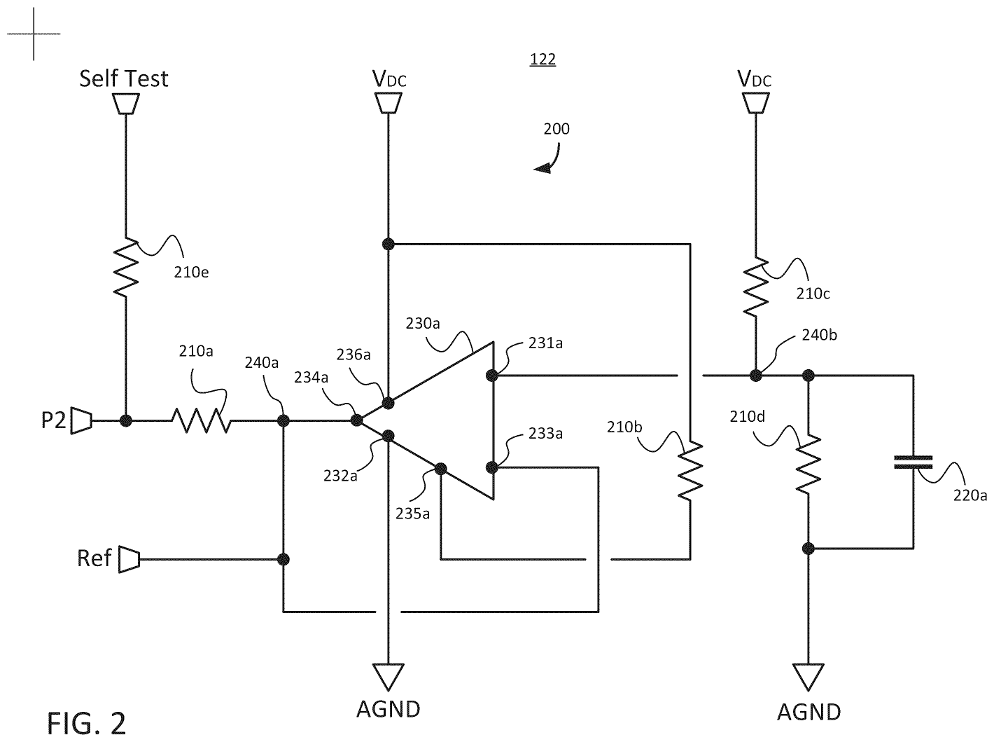

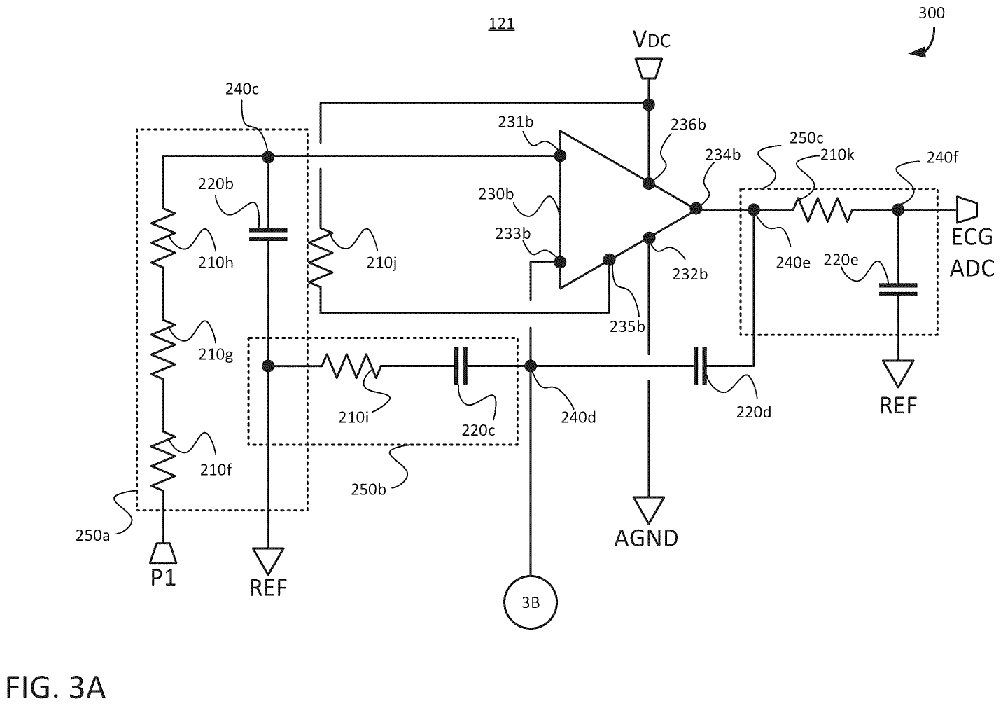

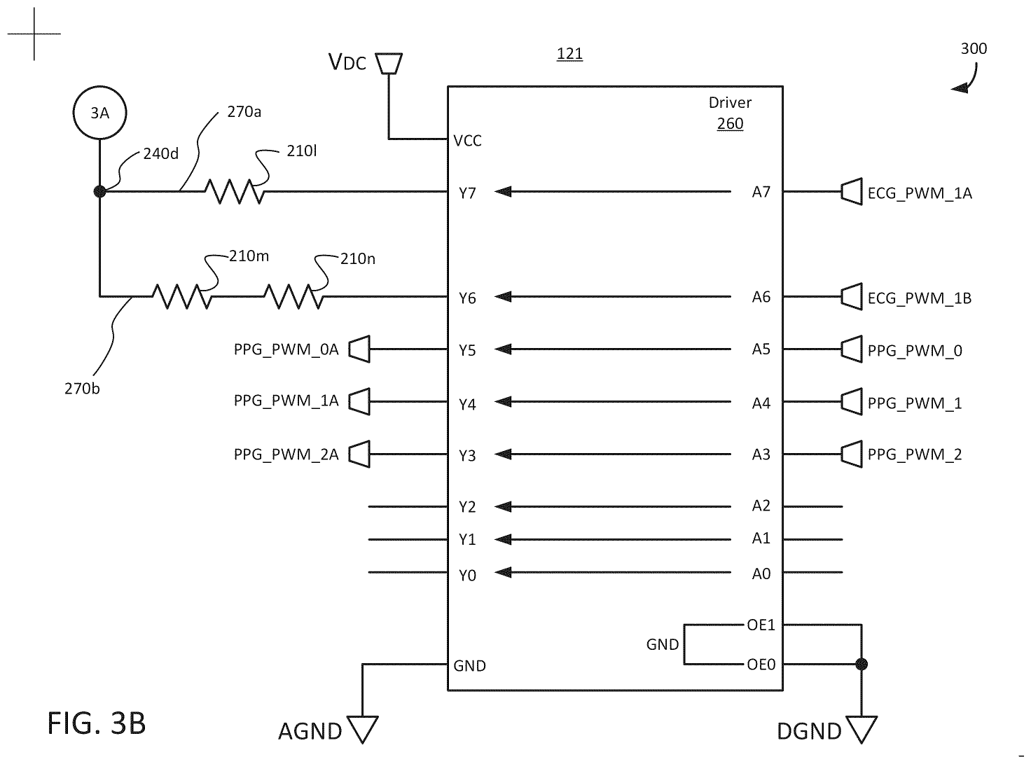

As is discussed in greater detail in regards to FIGS. 2, 3A, and 3B, the wearable monitor 120 may be understood as having two patch connections (200, 300) that operate together to provide the functionalities described herein. The first patch connection 300 is primarily the ECG analog front end, which receives an input from an electrode near or connected to the superior pole 121. The input is connected to an electrode near the superior pole 121. The output of the first patch connection 300 is filtered to produce a low frequency rejected ECG signal that goes to the Analog-to-Digital Converter in the Micro Control Unit (MCU). For example, ECG ADC at node 240f is the amplified output from the input from the first patch electrode P1, which is filtered by the ninth resistor 210i and the third capacitor 220c, the sixth through eighth resistors 210f-h and second capacitor 220b, and the eleventh resistor 210k and fifth capacitor 220e.

The second patch connection 200 describes the driving circuitry that drives the second electrode that is near or connected to the inferior pole 122. This driving circuitry drives the second electrode at the midpoint of the analog front end supply, and ultimately serves as a reference signal for the first patch connection 300.

Both patches may use the same battery and ground, and may be constructed on the same printed circuit board as one another, although the patches may be constructed separately. As part of self-testing, the MCU drives the second patch connection 200 with a predefined voltage/current pattern and that drives a signal pass through the body of a biological subject wearing the wearable monitor 120, which is sensed as part of the ECG signal through the first patch connection 300.

FIG. 2 is a circuit diagram of an example second patch connection 200 for an electrocardiogram (ECG) reader, according to embodiments of the present disclosure. In various embodiments, the second patch connection 200 is located in or in electrical communication with an inferior pole 122 of a wearable monitor 120, such as the example shown in FIG. 1, that is positioned inferior to or proximal to the xiphoid process of the subject 110 during use. In some embodiments, some or all of the electrical components of the second patch connection 200 are located in a monitor recorder 140 of the wearable monitor 120.

The second patch connection 200 is driven by a first operational amplifier (opamp) 230a (generally or collectively referred to with opamp 230b in FIG. 3A, as opamps 230). The opamps 230 include various pins for receiving or outputting various signals, which include, respectively, positive input pins 231a-b, ground pins 232a-b connected to respective grounds, inverting input pins 233a-b, output pins 234a-b, shutdown pins 235a-b; and supply voltage pins 236a-b. The first opamp 230a may be understood as a driving opamp 230, which drives the signals to the biological subject through the second patch connection P2 to bias the ECG signal from the biological subject during operation. This biased ECG signal is then sensed by the second opamp 230b via the first patch connection P1, and the second opamp 230b may be understood as a sensing opamp 230.

In FIG. 2, the first opamp 230a is connected with a first resistor 210a (generally or collectively with resistors 210a-l in FIGS. 2, 3A, and 3B, resistor 210) in series from the output pin 234a or the first node 240a to an ECG electrode P2 connected to the subject 100 during use. In various embodiments, the first resistor 210a defines a resistance of approximately 1 kilo-Ohm (kΩ). This low drive impedance reduces the possibility of electrostatic coupling into the distal electrode (e.g., the first patch electrode P1, discussed in relation to FIGS. 3A-3B, which is the input electrode that is high impedance and potentially sensitive to electrostatic coupling). By placing the electrodes under the monitor 120 and in contact with the subject 110, the design provides significant shielding to reduce electrostatic coupling.

A first node 240a (generally or collectively referred to with individual nodes 240a-d in FIGS. 2 and 3A-3B, as nodes 240) is disposed between the first resistor 210a and the output pin 234a. At the first node 240a, the ECG circuitry is referenced to a voltage that is half the regulated power supply voltage of V_{DC} from a power supply; REF. For example, when the power supply supplies a voltage of approximately 2.85 Volts (V), the REF voltage would be approximately 1.425 V. Because ECG electrodes can develop offsets on the order of 300 millivolts (mV), by using a reference voltage REF of 1.425V, the DC signal seen at the input ECG measurement electrode (in the first patch connection 300) is centered with approximately 1.1 V of excess margin compared to the supply voltage V_{DC}. Preferably, the first opamp 230a has very low power consumption with a 200 kilohertz (kHz) gain bandwidth that provides a low source impedance for the REF output to the ECG input gain amplifier. The first node 240a is connected to the inverting input pin 233a.

The power supply is connected directly to the supply voltage pin 236a and connected to the shutdown pin 235a via a second resistor 210b. In some embodiments, the second resistor 210b may be omitted to provide a 0Ω or direct connection, or may be replaced by a switch or transistor to controllably provide (e.g., via a gate voltage) an open circuit or direct connection between the power supply and the shutdown pin 235a. In various embodiments, the power supply is a battery, which may be rechargeable or single use. However, as the wearable monitor 120 is intended for sustained use with the subject 110, the present disclosure contemplates that extending the battery life of such a power supply is generally preferable to having to recharge or replace the power supply, but that such actions may also be performed.

As shown in FIG. 2, a second node 240b (e.g., a feedback node) that is connected directly with the positive input pin 231a is connected to the voltage source of VDC via a third resistor 210c, and to ground via a fourth resistor 210d and a first capacitor 220a (generally or collectively with capacitors 220a-e in FIGS. 2 and 3A, capacitor 220) in parallel to one another. In some embodiments, the third resistor 210c and fourth resistor 210d have substantially equal resistances (e.g., both 511 kΩ), and are used to set the reference voltage without adding significant current consumption on the power source. For example, the third resistor 210c and the fourth resistor 210d may add less than 3 micro-Amps (μA) of current consumption from a battery. The first capacitor 220a connected to AGND provides a low noise AGND reference to the REF voltage at half the supply voltage V_{DC}. In some embodiments, the first capacitor 220a has a capacitance of approximately 10 micro-Farads (μF).

The fifth resistor 210e with high resistance (e.g., having a resistance between 1 kΩ to 1 MΩ) acts as a resistive divider connected between a general purpose input/output (GPIO) pin (e.g., a self-test electrode) on the MCU and the first resistor 210a provides the ability to perform a self-test of the ECG circuitry. The resistive divider, when used, injects a transition in the voltage at the subject relative to REF (e.g., V_{DC}/10). This injection includes the subjection connection P2, and verifies that patch is properly connected to the subject 110, and that both the connection is good and that the circuitry is accurately measuring the ECG.

FIGS. 3A and 3B are circuit diagrams of an example first patch connection 300 for an ECG reader with an Analog to Digital Converter (ADC) therein, according to embodiments of the present disclosure. In various embodiments, the first patch connection 300 is located in or in electrical communication with a superior pole 121 of a wearable monitor 120, such as the example shown in FIG. 1, that is positioned superior to the xiphoid process of the subject 110 and inferior to or proximal to the manubrium of the subject 110. In some embodiments, some or all of the electrical components of the first patch connection 300 are located in a monitor recorder 140 of the wearable monitor 120.

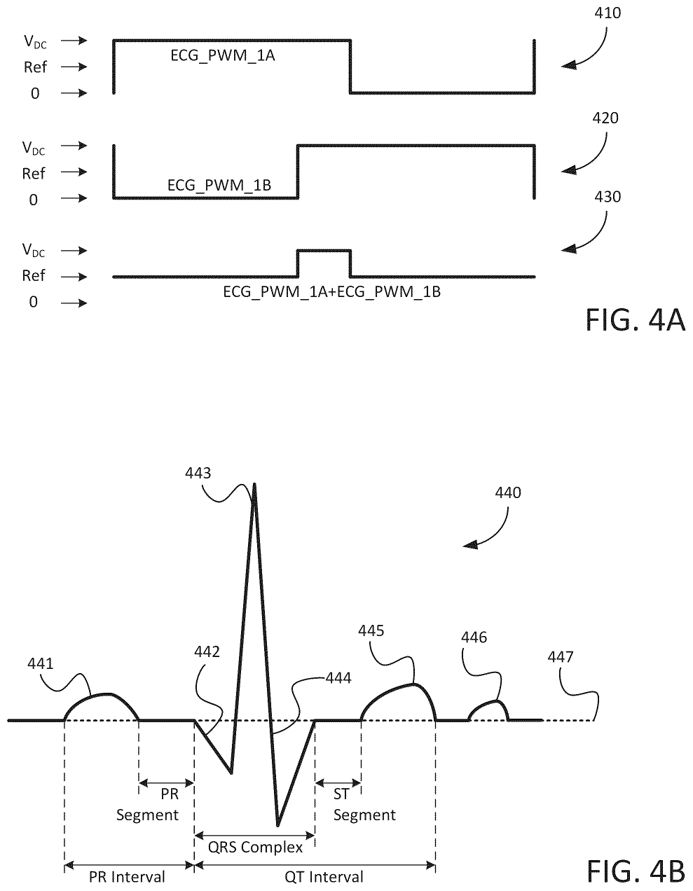

In FIG. 3A, the second opamp 230b is connected to an ECG electrode P1 connected to the subject 100 during use by at least one resistor 210 via the positive input pin 231b, and to a Pulse Width Modulated (PWM) signal source (discussed in greater detail in regard to FIGS. 3B and 4B) via the inverting input pin 233b. The output pin 234b of the second opamp 230b is connected to an output node 240f (e.g., ECGADC) via an eleventh resistor 210k. A fifth capacitor 220e is connected between the output node 240f and a reference voltage, a fourth capacitor 220d is connected between the output pin 234b and the inverting input pin 233b, and a third capacitor 220c in series with a ninth resistor 210i is connected between the inverting input pin 233b and the reference voltage. In various embodiments, a voltage supply (e.g., V_{DC}) is connected directly to a supply voltage pin 236b of the second opamp 230b and connected to a shutdown pin of the operational amplifier 230b via a resistor 210j, transistor, or switch, while the ground pin 232b of the second opamp 230b is connected to a ground.

The first patch connection 300 is connected to the P1 electrode, which is the input ECG signal relative to the P2 electrode (discussed in relation to FIG. 2), which is driven at the REF potential. The ECG signal from the P1 electrode is filtered by a first low pass filter (LPF) 250a (generally or collectively with LPFs 250a-c, LPF 250) of a resistive-capacitive (RC) LPF 250 with a corner frequency of approximately 1.6 kHz. As illustrated, the first low pass filter 250a uses three high voltage resistors 210 (e.g., resistors 210f-210h) to protect against defibrillation of a subject that is wearing the monitor 120, although two or more of these resistors 210 may be combined into a single resistor 210 or the three resistors 210 may be divided into further discrete resistors 210 in various embodiments. The additional resistance from these low pass filter resistors 210 only adds a minor amount of noise (e.g., 650 nVpp of noise in a 25 Hertz (Hz) bandwidth when using three 10 kΩ resistors 210 for 30 kΩ series resistance). In contrast, the input impedance of the second opamp 230b is very high, with preferably less than 35 pico-amps (pA) of input bias current and 3.5 pF of input capacitance. Therefore, the input impedance of the ECG system is dominated (e.g., determined primarily by) by the capacitance of the second capacitor 220b, which in some embodiments is 33 nF.

The second opamp 230b amplifies the ECG signal prior to digitization. The single gain stage of the second opamp 230b reduces power consumption and improves noise performance of the analog ECG circuitry compared to multi-stage amplifiers. The gain of the second opamp 230b is set via the capacitances of the third capacitor 220c and the fourth capacitor 220d. For example, the second opamp 230b provides a gain of approximately 450× when the capacitances of the third capacitor 220c and the fourth capacitor 220d are set to approximately 100 μF and 220 nF, respectively. As will be appreciated, the precision for the set gain for the second opamp 230b is affected by the precision of the capacitance values of the third capacitor 220c and the fourth capacitor 220d, which can be calibrated at the time of manufacture and/or calibrated during self-test when first connected to the subject with the GPIO signal applied to the P2 electrode (as discussed in relation to FIG. 2).

In the first patch connection 300, are three LPF 250a-c built into this single opamp gain stage. The first LPF 250a is provided by the resistors 210f-h and second capacitor 220b at the P1 electrode/subject connection. The contact impedance of the P1 electrode adds to the input resistance of the first LPF 250a, and therefore the cutoff frequency of the first LPF 250a is affected by the impedance of the subject.

The second LPF 250b is set by the resistance of the ninth resistor 210i (located in series with the third capacitor 220c) and the capacitance of the third capacitor 220c. In some embodiments, the resistance of the ninth resistor 210i is approximately 20Ω and the capacitance of the third capacitor 220c is approximately 100 μF, which yields a corner frequency for the second LPF 250b of 81 Hz. Accordingly, with a 100 kHz gain bandwidth of the second opamp 230b, the gain at 81 Hz would be 1235× in this example. The second opamp 230b would therefore have an excess gain of 2.84× (1235/454=2.84). This second LPF 250b also keeps the second opamp 230b in a state where there is excess gain in the feedback loop of the second opamp 230b for the frequencies above 81 Hz.

The third LPF 250c is set by the resistance of the eleventh resistor 210k and the capacitance of the fifth capacitor 220e. In some embodiments, the resistance of the eleventh resistor 210k is set to approximately 51 kΩ and the capacitance of the fifth capacitor 220e is set to approximately 3.3 nF, which results in the third LPF 250c having a corner frequency of approximately 1 kHz.

The second opamp 230b, in addition to using capacitors 220 to set the gain thereof, uses a pulse width modulated (PWM) feedback signal applied to the inverting input pin 233b. PWM feedback signals are used because such signals are readily available on an MCU. Digital to Analog conversion of PWM signals is very linear, such that a single step in PWM value produces a precise step in analog voltage thanks to the repeatable precise timing of the MCU crystal clock source, and with high valued resistors 210, the PWM power consumption is very low, among other benefits.

In order to reduce the likelihood of (or prevent) noise on the MCU digital power supply affecting the PWM signal, a driver chip 260 (as shown in FIG. 3B) is used to drive the PWM signal using the low noise regulated analog signal from the power source. In some embodiments, the magnitude of the power signal V_{DC} is 2.85 V. The noise of the PWM at the inverting pin 233b is a combination of the resistor noise plus the power supply noise.

For example, a 258 kΩ PWM source resistance that has 64 nV/rtHz of noise, which translates to 1.9 μV peak-to-peak noise in a 25 Hz bandwidth. However, the 258 kΩ resistance is low pass filtered with the third capacitor 220c (e.g., of 10 μF) so that this resistor noise source is reduced by a factor of 10 at the low frequency corner of the ECG bandwidth of 0.65 Hz-making this noise source small compared to the noise of the second opamp 230b. Continuing the example, when using a power supply with a regulated power supply of about 4 μV/rtHz at 10 Hz, the noise translates to 27 nV/rtHz at 10 Hz after the low pass filter of the 258 kΩ and 10 μF input to the second opamp 230b, so the regulator noise at 10 Hz is also less than the opamp source noise.

However, due to the risk of unspecified noise for the regulator producing a low frequency baseline wander down, for example in the 0.65 to 2 Hz region, there are several possible mitigation strategies that can be applied. One mitigation strategy is to increase the PWM resistance, for example by introducing slew at 125 mV/s. A PWM source resistance as high as 1 MΩ2 can still slew the signal at the second opamp 230b with 125 mV/s from the PWM, which would increase the LPF rejection of regulator noise by a factor of 4×. Additionally or alternatively, a different regulator or add additional filtering can be used with the regulated voltage supply.

The driver chip 260 receives various signals, including ECG_PWM_1A, ECG_PWM_1B, an input power from the power supply of V_{DC}/V_{CC}, and is also connected to ground (e.g., AGND and DGND). In various embodiments, the driver chip 260 collects photoplethysmogram (PPG) signals are related to cardiac changes in blood volume per heartbeat, (e.g., PPG_PWM_0, PPG_PWM_1, and PPG_PWM_2) as well as other signals collected by the monitor 120. Each of these signals is received as a digital signal by the driver chip 260 at an input pin, and converted into a filtered digital signal output via a corresponding output pin. In various embodiments, one or more pins of the driver chip 260 may be unused, or designated for additional purposes to those given in the present examples.

The average voltage of the PWM signal of the LPF 250b is filtered by the third capacitor 220c, and is compared with the incoming signal from the P1 electrode. When the incoming signal from the P1 electrode is greater than the average PWM signal, the ECG_ADC output from the second opamp 230b increases. The increase of the ECG_ADC output is used to feedback an increase in the PWM signal to cancel the increased signal from the subject. The PWM signal is sourced from the MCU, and is applied at the fourth node 240d. The delay of the feedback may be two sample periods of the ADC sample rate; one sample period for sensing that a new PWM is set and a second sample period to wait for the next clock signal. For example, with a sample rate of 327 Hz, a stable loop bandwidth of 40 Hz can be easily achieved since this delay will only result in an added 90 degrees of phase shift in the feedback signal when using a two sample delay. This PWM signal is filtered with a low pass corner frequency set by the ninth resistor 210i and the third capacitor 220c (e.g., for value of approximately 0.062 Hz). To achieve the 40 Hz bandwidth, the ADC measured value is multiplied by 1.4 (40/(0.062*454)) for the appropriate PWM value to apply. With 40 Hz loop bandwidth, the PWM signal is dynamically cancelling more than the DC offset. In fact, the PWM signal is substantially reducing the amplitude of the ECG signal that is digitized at the ADC at a rate of 20 dB per decade for frequencies less than 40 Hz.

For example, a 10 mV peak to valley signal at 10 Hz will be reduced to an amplitude of 2.5 mV peak-to-valley with a closed loop bandwidth of 40 Hz for the PWM. Without this feedback, the ECG dynamic range at a gain of 454 would be 2.85/454=6 mV peak-to-valley. With the feedback, the ECG dynamic range is 24 mV peak-to-valley at 10 Hz which exceeds the 10 mV as specified by the standards. The full DC coupled ECG signal is calculated in the MCU by applying a digital filter to the PWM value that matches the analog filter due to the PWM resistors and the third capacitor 220c. Since this same signal is subtracted at the second opamp 230b, this signal is added back into the digitized signal with the known delay of one full ADC sample to thereby produce a full DC coupled ECG measurement over the desired ECG bandwidth.

The result of this PWM feedback method is to increase the effective number of bits of the ADC by an additional 8 bits at DC. So a 12-bit ADC at the MCU will effectively become a 20-bit ADC at DC levels. The least significant bit (LSB) bit resolution remains constant over the range from DC to 40 Hz, but the dynamic range advantageously decreases linearly as a function of voltage relative to frequency (e.g., ±1.4V at 0.063 Hz, ±140 mV at 0.63 Hz, ±14 mV at 6.3 Hz, etc.). With ECG measurement, one challenge is to tolerate large low frequency variations while measuring a small ECG signal with low noise. This PWM method advantageously matches the needs of an ECG measurement system.

Additionally, the DC offset of the ECG can be important for designers and clinicians to know. For example, a large DC offset of the ECG electrode could imply a failure of the electrode (e.g., due to manufacturing defects or an electrode that is beyond shelf life). When coupled with a subject that has a noisy ECG, the large DC offset gives the factory engineer additional information as to why there might be excessive noise. Additionally, the DC offset can be available for users to troubleshoot poor ECG quality.

FIG. 4A illustrates example digital signals used by the ADC described herein, on which the x-axis represents time, and the y-axis represents voltage at one of three levels (e.g., V_{DC}, Ref, and 0 Volts).

The measurement device described herein sums together two digital PWM signals (shown as ECG_PWM_1A and ECG_PWM_1B with respect to FIGS. 3A and 3B). ECG_PWM_1A and ECG_PWM_1B are provided from a microcontroller, and have the same frequency but different duty cycles from one another. The two digital PWM signals sum together at the fourth node 240d (See, FIG. 3B). The summed average over a cycle is the targeted low frequency rejection level (Vfb), which may be calculated by: obtaining feedback factor (B), according to the desired passband (Fₚ) divided by the product of band of rejected low frequency (Fr) and the system gain (G), according to Formula 1, and multiplying the feedback factor by the bandwidth of the ADC according to Formula 2

$$β = {F}_{p} / ( {F}_{r} * G ) \qquad Formula 1$$

$${V}_{fb} = β * ADC \qquad Formula 2$$

Accordingly, the DC offset of the subject connection is zero, the first signal 410 (e.g., ECG_PWM_1A) is the inverse of the second signal 420 (e.g., ECG_PWM_1B), so that the summed signal 430 that is provided into the inverting input pin 233b of the second opamp 230b is equivalent to REF. The current from the PWM source is integrated at the fourth capacitor 220d. If only one PWM were used in this application, the opamp output at the fifth node 240e (e.g., at the fourth capacitor 220f) would result in a triangle wave that is relatively large, which would negatively affect the dynamic range of the second opamp 230b and thereby create a scenario where the second opamp 230b is ramping at the time where the ADC makes a measurement near the end of the PWM cycle. With two PWM drive signals 410, 420, the second opamp 230b has a minimal ramp signal that is a function of the DC offset of the subject voltage.

For example, FIG. 4A shows a case where the ECG_PWM_1A signal 410 is high for more than half the cycle and the ECG_PWM_1B signal 420 is high for less than half the cycle. When summed together, the combined signal 430 is at REF for most the time with a short pulse at the center of the PWM cycle to cancel a small offset in voltage coming from the subject, which eliminates the constant ramping created by a single PWM, and produces only a small shift in DC level at the center of the PWM cycle.

When combining two PWM signals for use as a feedback signal, the resolution can be increased compared to using a single PWM signal. For example, the resolution of a 652 Hz PWM period with a 65.2 kHz clock is only 200 counts; however, when two PWMs using the same clock are combined together, the resolution is increased to 400 counts. This additional resolution is achieved via the addition of the fourteenth resistor 210n to one signal pathway 270a-b (as shown in FIG. 3B) for one of the first digitized signal 410 or second digitized signal 430 (for ECG_PWM_1A and ECG_PWM_1B, respectively). Although illustrated in FIG. 3B in series with the thirteenth resistor 210m on the second signal pathway 270b, in some embodiments the fourteenth resistor 210n may instead be placed in series with the twelfth resistor 210l on the first signal pathway 270a to also achieve this effect. Generally, the resistances of the twelfth and thirteenth resistors 210l-m are substantially equal to one another, and greater than the fourteenth resistor 210n, where the resistance ratio of the twelfth/thirteenth resistor 210l-m to the fourteenth resistor 210n provides a corresponding increase in resolution. For example, the twelfth and thirteenth resistors 210l-m may have resistances of about 1 MΩ, while the fourteenth resistor 210n is 1/50 of that value (e.g., about 20 kΩ), resulting in an additional resolution factor of 50×; thereby resulting in a total resolution of 10,000 (e.g., 200×50).

FIG. 4B illustrates components of an example ECG waveform 440, on which the x-axis represents time, and the y-axis represents cutaneous electrical signal strength relative to a baseline 447. The P-wave 441 has a smooth, normally upward, that is, positive, waveform that indicates atrial depolarization. The QRS complex often begins with the downward deflection of a Q-wave 442, followed by a larger upward deflection of an R-wave 443, and terminated with a downward waveform of the S-wave 444, collectively representative of ventricular depolarization. The T-wave 445 is normally a modest upward waveform, representative of ventricular depolarization, while the U-wave 446, often not directly observable, indicates the recovery period of the Purkinje conduction fibers.

Sampling of the intervals between successive waveforms 440 enables heart rate information derivation. For instance, the R-to-R interval represents the ventricular rate and rhythm, while the P-to-P interval represents the atrial rate and rhythm. Importantly, the PR interval is indicative of atrioventricular (AV) conduction time and abnormalities in the PR interval can reveal underlying heart disorders, thus representing another reason why the P-wave quality achievable by the ambulatory electrocardiography monitoring patch optimized for capturing low amplitude cardiac action potential propagation described herein is medically unique and important. The long-term observation of these ECG indicia, as provided through extended wear of the wearable monitor 120, provides valuable insights to the subject's cardiac function symptoms, and overall well-being, which may be used for the treatment or prophylaxis of various conditions.

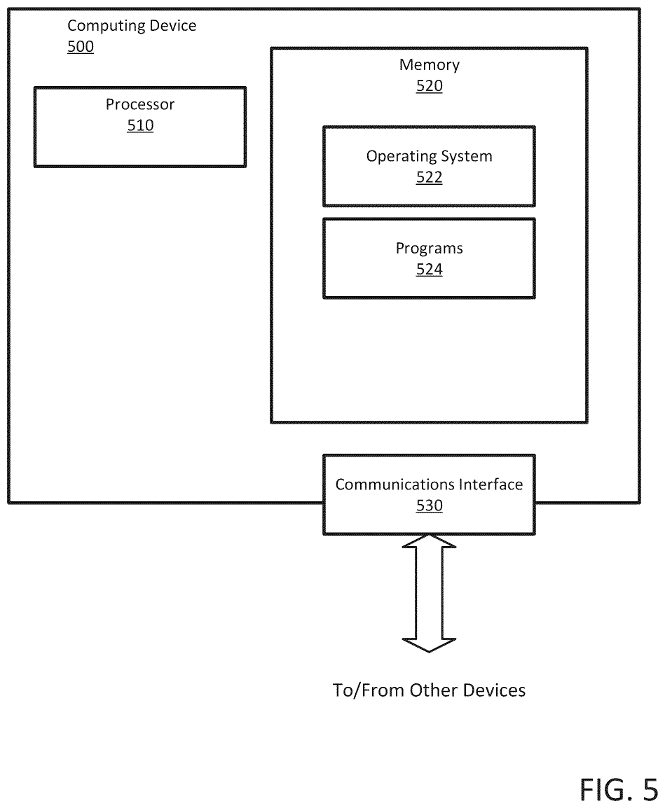

FIG. 5 illustrates a computing device 500, as may be used as an MCU to collect, collate, and output digital measurements of an ECG signal to produce an ECG, according to embodiments of the present disclosure. The computing device 500 may include at least one processor 510, a memory 520, and a communication interface 530.

The processor 510 may be any processing unit capable of performing the operations and procedures described in the present disclosure. In various embodiments, the processor 510 can represent a single processor, multiple processors, a processor with multiple cores, and combinations thereof.

The memory 520 is an apparatus that may be either volatile or non-volatile memory and may include RAM, flash, cache, disk drives, and other computer readable memory storage devices. Although shown as a single entity, the memory 520 may be divided into different memory storage elements such as RAM and one or more hard disk drives. As used herein, the memory 520 is an example of a device that includes computer-readable storage media, and is not to be interpreted as transmission media or signals per se.

As shown, the memory 520 includes various instructions that are executable by the processor 510 to provide an operating system 522 to manage various features of the computing device 500 and one or more programs 524 to provide various functionalities to users of the computing device 500, which include one or more of the features and functionalities described in the present disclosure. One of ordinary skill in the relevant art will recognize that different approaches can be taken in selecting or designing a program 524 to perform the operations described herein, including choice of programming language, the operating system 522 used by the computing device 500, and the architecture of the processor 510 and memory 520. Accordingly, the person of ordinary skill in the relevant art will be able to select or design an appropriate program 524 based on the details provided in the present disclosure.

The communication interface 530 facilitates communications between the computing device 500 and other devices, which may also be computing devices as described in relation to FIG. 5. In various embodiments, the communication interface 530 includes antennas for wireless communications and various wired communication ports. The computing device 500 may also include or be in communication, via the communication interface 530, one or more input devices (e.g., a keyboard, mouse, pen, touch input device, etc.) and one or more output devices (e.g., a display, speakers, a printer, etc.). The computing device 500 may be in communication with one or more of the second patch connection 200 and the first patch connection 300.

Although not explicitly shown in FIG. 5, it should be recognized that the computing device 500 may be connected to one or more public and/or private networks via appropriate network connections via the communication interface 530. It will also be recognized that software instructions may also be loaded into a non-transitory computer readable medium such as the memory 520 from an appropriate storage medium or via wired or wireless means.

Accordingly, the computing device 500 is an example of a system that includes a processor 510 and a memory 520 that includes instructions that (when executed by the processor 510) perform various embodiments of the present disclosure. Similarly, the memory 520 is an apparatus that includes instructions that when executed by a processor 510 perform various embodiments of the present disclosure.

Certain terms are used throughout the description and claims to refer to particular features or components. As one skilled in the art will appreciate, different persons may refer to the same feature or component by different names. This document does not intend to distinguish between components or features that differ in name but not function.

As used herein, various units of measure may be referred to by associated short forms with various prefixes applied thereto as set by the International System of Units (SI), which one of ordinary skill in the relevant art will be familiar with.

As used herein, “about,” “approximately” and “substantially” are understood to refer to numbers in a range of the referenced number, for example the range of −10% to ±10% of the referenced number, preferably −5% to ±5% of the referenced number, more preferably −1% to ±1% of the referenced number, most preferably −0.1% to ±0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the present description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

While the invention has been particularly shown and described as referenced to the embodiments thereof, those skilled in the art will understand that the foregoing and other changes in form and detail may be made therein without departing from the spirit and scope.

What is claimed is:

1. An analog to digital (A2D) converter for an electrocardiogram (ECG) measuring device, comprising:
a voltage supply;
an operational amplifier, including:
a positive input pin;
an inverting input pin; and
an output pin;
an output node, connected directly to the output pin and the inverting input pin;
an ECG electrode, connected to the output node via a first resistor;
a reference voltage, connected directly to the output node; and
a feedback node, connected to directly to the positive input pin, connected to the voltage supply via a second resistor, and connected to a ground via a third resistor and a first capacitor, wherein the third resistor and the first capacitor are connected in parallel between the feedback node and the ground.

2. The A2D converter of claim 1, further comprising a self-test electrode connected to the first resistor via a fifth resistor.

3. The A2D converter of claim 1, wherein the operational amplifier further includes a supply voltage pin, a shutdown pin, and a ground pin that is connected to the ground, further comprising:
a voltage supply, connected directly to the supply voltage pin and connected to the shutdown pin via a second resistor.

4. The A2D converter of claim 1, further comprising:
a second operational amplifier, including:
a second positive input pin;
a second inverting input pin; and
a second output pin;
a second output node, connected directly between a fifth resistor connected to the second output pin and connected to a second capacitor that is connected to the reference voltage;
a second ECG electrode, connected to the second positive input pin via at least one resistor;
a third capacitor connected to the second positive input pin and the reference voltage;
a sixth resistor and a fourth capacitor connected in series between the reference voltage and the second inverting input pin;
a fifth capacitor connected between the second inverting input pin and the second output pin; and
a Pulse Wave Modulated (PWM) signal source connected to the second inverting input pin.

5. An analog to digital (A2D) converter for an electrocardiogram (ECG) measuring device, comprising:
an operational amplifier, including:
a positive input pin;
an inverting input pin; and
an output pin;
an output node, connected directly between a first resistor connected to the output pin and connected to a first capacitor that is connected to a reference voltage;
an ECG electrode, connected to the positive input pin via at least one resistor;
a second capacitor connected to the positive input pin and the reference voltage;
a second resistor and a third capacitor connected in series between the reference voltage and the inverting input pin;
a fourth capacitor connected between the inverting input pin and the output pin; and
a Pulse Wave Modulated (PWM) signal source connected to the inverting input pin.

6. The A2D converter of claim 5, wherein the operational amplifier further includes a supply voltage pin, a shutdown pin, and a ground pin that is connected to a ground, further comprising:
a voltage supply, connected directly to the supply voltage pin and connected to the shutdown pin via a second resistor.

7. The A2D converter of claim 5, wherein the at least one resistor and the second capacitor define a low pass filter with a corner frequency of approximately 1.6 kilohertz.

8. The A2D converter of claim 5, wherein the second resistor and the third capacitor define a low pass filter with a corner frequency of approximately 81 Hertz.

9. The A2D converter of claim 5, wherein the first resistor and the first capacitor define a low pass filter with a corner frequency of approximately 1 kilohertz.

10. The A2D converter of claim 5, wherein the PWM signal source combines a first digital PWM signal with a second digital PWM signal before delivering a combined PWM signal to the inverting input pin.

11. The A2D converter of claim 5, further comprising:
a voltage supply;
a second operational amplifier, including:
a second positive input pin;
a second inverting input pin; and
a second output pin;
an output node, connected directly to the output pin and the inverting input pin;
a second ECG electrode, connected to the output node via a third resistor;
a reference electrode, connected directly to the output node;
a feedback node, connected to directly to the positive input pin, connected to the voltage supply via a third resistor, and connected to a ground via a fourth resistor and a first capacitor, wherein the fourth resistor and the first capacitor are connected in parallel between the feedback node and the ground.

12. The A2D converter of claim 11, further comprising a self-test electrode connected to the first resistor via a fifth resistor.

13. An analog to digital (A2D) converter for an electrocardiogram (ECG) measuring device, comprising:
an operational amplifier;
a first low pass filter, connected between an ECG electrode, a reference voltage, and a positive input pin of the operational amplifier;
a second low pass filter, connected between the reference voltage and an inverting input of the operational amplifier;
a third low pass filter, connected between an output pin of the operational amplifier, the reference voltage, and a signal output;
a first signal pathway connected between a first digital pulse wave modulated (PWM) signal source and the inverting input of the operational amplifier; and
a second signal pathway connected between a second digital PWM signal source and the inverting input of the operational amplifier, wherein the second signal pathway combines with the first signal pathway before connecting with the first signal pathway.

14. The A2D converter of claim 13, wherein the first low pass filter has a corner frequency of approximately 1.6 kilohertz and includes a series of resistors disposed between the ECG electrode and the positive input pin and a capacitor disposed between the positive input pin and the reference voltage.

15. The A2D converter of claim 13, wherein the second low pass filter has a corner frequency of approximately 81 Hertz and includes a resistor connected between the reference voltage and a capacitor, wherein the capacitor is connected between the resistor and the inverting input of the operational amplifier.

16. The A2D converter of claim 13, wherein the third low pass filter has a corner frequency of approximately 1 kilohertz and includes a resistor connected between the output pin of the operational amplifier and the signal output and include a capacitor is connected between the signal output and the reference voltage.

17. The A2D converter of claim 13, wherein the first signal pathway has a first resistance, and the second signal pathway has a second resistance, greater than the first resistance, as measured between respective pins of a driver chip and a node shared with the inverting input of the operational amplifier.

18. The A2D converter of claim 13, further comprising:
a voltage supply, connected directly to a supply voltage pin of the operational amplifier and connected to a shutdown pin of the operational amplifier via a resistor, transistor, or switch.

19. The A2D converter of claim 18, further comprising:
a second operational amplifier, including:
a second positive input pin;
a second inverting input pin; and
a second output pin;
a second output node, connected directly to the second output pin and the second inverting input pin;
a second ECG electrode, connected to the second output node via a first resistor;
a reference electrode, connected directly to the second output node;
a feedback node, connected directly to the second positive input pin, connected to the voltage supply via a second resistor, and connected to ground via a third resistor and a first capacitor, wherein the third resistor and the first capacitor are connected in parallel between the feedback node and the ground.

20. The A2D converter of claim 19, further comprising a self-test electrode connected to the first resistor via a fifth resistor.
